(19) United States
(12) Patent Application Publication
(10) Pub. No.: US 2014/0080086 A1
(43) Pub. Date: Mar. 20, 2014

(54) Image Navigation Integrated Dental Implant System

(71) Applicant: Roger CHEN, New Taipei City (TW)

(72) Inventor: Roger CHEN, New Taipei City (TW)

(21) Appl. No.: 13/623,586

(22) Filed: Sep. 20, 2012

(51) Int. Cl. A61B 6/14

(52) U.S. Cl. 433/29

(57) ABSTRACT

An image navigation integrated dental implant system includes a control unit for storing and transmitting data streams, a scan module electrically connected to the control unit for scanning and taking the pictures of the soft tissue and hard tissue of the parenchyma of the oral cavity of a patient and the related external skin color and transmitting the obtained data to the control unit, a design module electrically connected to the control unit for receiving the oral cavity data from the control unit and using the data to design a oral cavity simulation diagram, and a navigator module electrically connected to the control unit for receiving the oral cavity data and photographing the oral cavity and then providing a picture to guide the dentist to perform the dental implant surgery.

### BACKGROUND OF THE INVENTION

1. Field of the Invention

The present invention relates to medical implements and more particularly, to an image navigation integrated dental implant system, which integrates oral cavity scanning, mold-making and surgery navigation into one system, thereby shortening operation time and improving the accuracy of the surgery.

2. Description of the Related Art

The evolution and advancement of dental implant surgery is a great boon to people suffering dental problems. Employing a surgery to make and implant an artificial tooth can help the patient have a face-lift and also improve the quality of the patient's life.

A dental implant surgery is to install an artificial tooth root in the gum tissue of the jaw bone, and then to fasten an artificial teeth to the artificial tooth root. Early techniques emphasize the assessment of bone quality, determining the suitability of the patient's bone quality for artificial tooth root implantation. However, during assessment, teeth bite condition may be neglected. If a significant axial offset of the implant occurs after the dental implant surgery, the service life of the implant will be shortened even a correction is made, and fusion between the jaw bone and the implant may fail a certain period of time after the dental implantation.

Therefore, in order to avoid the problems of the aforementioned known practice, computer tomography (CT) together with auxiliary computer software diagnosis are employed, enabling the medical persons to see the structure of the bones and the distribution of important nerves when making a model, and helping the dentist assess the bone quality and tooth bite condition of the patient. Thus, the dentist can simulate the surgery on the computer to avoid touching important nerves and blood vessels in the implant surgery, greatly improving surgical safety, and giving better protection to the patient and physician.

An implant planning prior to dental surgery relies on the use of tomography to establish an oral model. Based on the established oral model, the dentist can make a better implant planning. In order to perform a dental surgery accurately, implant surgical guide plates shall be made prior to the dental implant surgery. Subject to the assistance of the prepared implant surgical guide plates and a computer navigation system, the dentist can perform the surgery in the patient's oral cavity accurately.

The technique of using implant surgical guide plates to assist a dental implant surgery is quite mature. However, the preparation of implant surgical guide plates takes much time. For general clinics, it needs to scan the patient's oral cavity in order to establish an oral model data, and then to send the scanned oral model data to a guide plate making center at another place. The round-trip time is quite time-consuming. Further, if any produced guide plate is defective unable to be timely corrected, much time can be wasted while waiting for the correct guide plate.

### SUMMARY OF THE INVENTION

The present invention has been accomplished under the circumstances in view. It is one object of the present invention to provide an image navigation integrated dental implant system, which integrates oral cavity scanning, mold-making and surgery navigation into one system, thereby shortening operation time and improving the accuracy of the surgery.

To achieve this and other objects of the present invention, an image navigation integrated dental implant system comprises a control unit, a scan module, a design module and a navigator module. The control unit is adapted for integrating the storage and transmission of data streams. The scan module is electrically connected to the control unit. The scan module comprises a scan unit and a picture-taking unit, and is adapted to scan and to take the pictures of the soft tissue and hard tissue of the parenchyma of the oral cavity of a patient and the external skin color of the oral cavity of the patient and then to transmit the obtained data to the control unit. The design module is electrically connected to the control unit, and adapted to receive the oval cavity data of the patient from the control unit and to design related dental molds using the received oval cavity data of the patient. The design module comprises a design unit and a mold-making unit electrically connected together. The design unit is adapted to design an oral cavity simulation diagram using the oral cavity data. The mold-making unit is adapted to make an oral model, a tooth mold and implant surgical guide plates subject to the oral cavity simulation diagram. The navigator module is electrically connected to the control unit, and adapted to receive the oral cavity data from the control unit. The navigator module comprises a display unit and a photograph unit. The photograph unit is electrically connected to the display unit, and adapted to convert images of the relative position between the oral cavity and the loaded implant surgical guide plates into corresponding electrical image signals and to transmit the electrical image signals to the display unit for generating a picture for enabling the generated picture be overlapped on the displayed simulation picture to help the dentist perform the surgery.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is a system block diagram of an image navigation integrated dental implant system in accordance with the present invention.

FIG. 2 is a flow chart of a dental implant surgery using the image navigation integrated dental implant system in accordance with the present invention.

### DETAILED DESCRIPTION OF THE PREFERRED EMBODIMENT

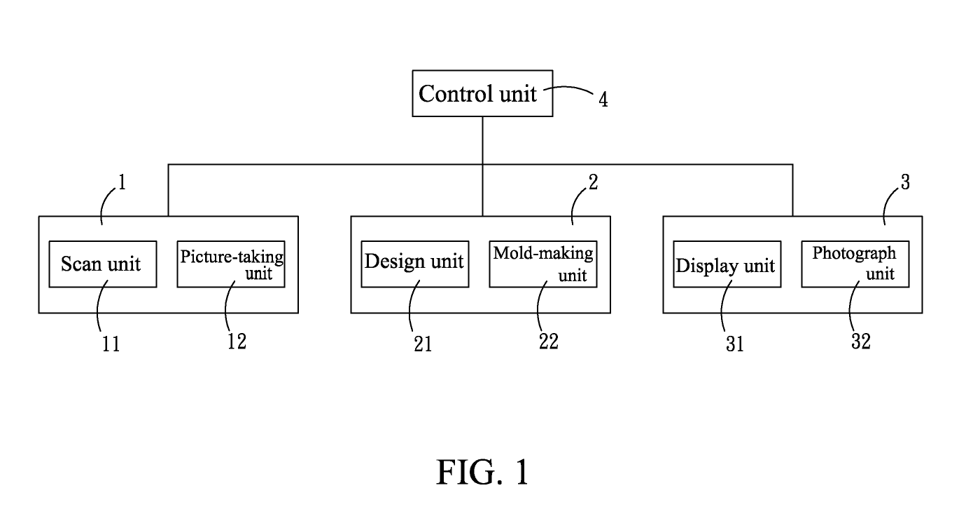

Referring to FIG. 1, an image navigation integrated dental implant system in accordance with the present invention is shown comprising a scan module 1, a design module 2, a navigator module 3, and a control unit 4.

The control unit 4 is electrically connected with the scan module 1, the design module 2 and the navigator module 3, and adapted to control and transmit their output electronic signals and data streams. The scan module 1 is adapted to scan the inside of the mouth of the patient, including the soft tissue and hard tissue of the parenchyma and the external skin color of the oral cavity, and to provide the scanned data to the control unit 4. The scan module 1 comprises a scan unit 11 having at least one of the functions of full body scan, magnetic resonance imaging (MRI), ultrasound imaging, computer tomography and intra and extra oral scan in a “2D” mode or “3D” mode, and a picture-taking unit 12 that is a color camera capable of taking color pictures. The design module 2 is electrically connected to the control unit 4, and adapted to receive data streams from the control unit 4 and to make molds subject to the received data streams. The design module 2 comprises a design unit 21 and a mold-making unit 22. The design unit 21 is adapted to receive scanned oral cavity data from the control unit 4, and to design an oral cavity simulation diagram in a 2D or 3D mode using the scanned oral cavity data. The design unit 21 is a design software, for example CAD/CAM (computer-aided design/computer-aided manufacturing) design software. The mold-making unit 22 is electrically connected to the design unit 21, and adapted to receive the data of the oral cavity simulation diagram designed by the design unit 21 and to make molds, including oral model, tooth mold and implant surgical guide plates, subject to the data of the oral cavity simulation diagram, In this embodiment, the mold-making unit 22 is a dental milling machine.

Referring to FIG. 1 again, the navigator module 3 is adapted to receive scanned oral cavity data from the control unit 4, comprising a display unit 31 and a photograph unit 32. The display unit 31 and the photograph unit 32 are electrically connected together. Further, the display unit 31 can be a display screen or in-goggle display device adapted to display images transmitted from the photograph unit 32. The photograph unit 32 is a video camera adapted to convert images of the relative position between the implant surgical guide plates and the oral cavity into electrical image signals and to transmit the electrical image signals to the display unit 31, enabling the display unit 31 to generate a picture and to overlap the generated picture on a displayed simulation picture for guiding the dentist to perform the implant surgery. Further, the control unit 4 is a computer adapted to integrate the storage and transmission of the data streams provided by all the modules of the image navigation integrated dental implant system, to establish a simulation picture using the scanned oral cavity data, and to transmit the established simulation picture to the display unit 31 for display, assisting the dentist to perform the dental implant surgery.

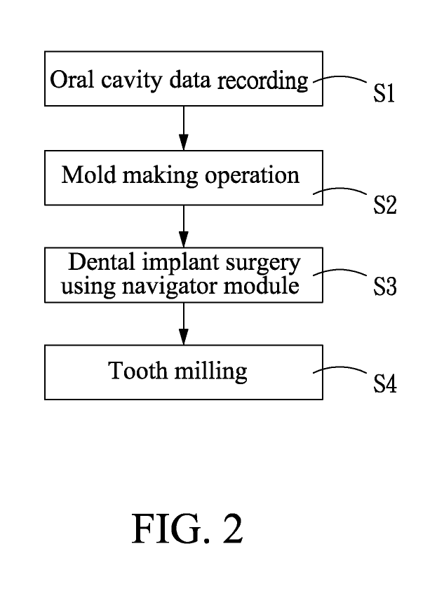

FIG. 2 is a flow chart of a dental implant surgery using the image navigation integrated dental implant system in accordance with the present invention. The dental implant surgery includes the following steps:

(S1) Oral cavity data recording: where the scan unit 11 and picture-taking unit 12 of the scan module 1 are controlled to scan and take the pictures of the inside of the mouth of the patient, recording the structure and outer appearance of the patient's oral cavity for making an initial diagnosis and plan prior to the surgery.

(S2) Mold making operation: where the design unit 21 and the mold-making unit 22 are controlled to simulate implant installation and to make an oral model, a tooth mold and implant surgical guide plates.

(S3) Dental implant surgery using navigator module: where, after the oral model, the tooth mold and the implant surgical guide plates are made, the display unit 31 is controlled to display simulation pictures, guiding the dentist to perform the surgery, enabling the dentist to control the progress and location of the implant procedure, presenting the dentist from damaging other nerves or bones in the oral cavity, and ensuring high accuracy of the surgery.

(S4) Tooth milling: where a tooth milling operation is started after the surgery, modifying the tooth form to the most beautiful status.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. An image navigation integrated dental implant system, comprising:
a control unit for integrating the storage and transmission of data streams;
a scan module electrically connected to said control unit, said scan module comprising a scan unit and a picture-taking unit and being adapted to scan and to take the pictures of the soft tissue and hard tissue of the parenchyma of the oral cavity of a patient and the external skin color of the oral cavity of said patient and then to transmit the obtained data to said control unit;
a design module electrically connected to said control unit and adapted to receive the oval cavity data of said patient from said control unit and to design related dental molds using the received oval cavity data of said patient, said design module comprising a design unit and a mold-making unit, said design unit and a mold-making unit being electrically connected together, said design unit being adapted to design an oral cavity simulation diagram using said oral cavity data, said mold-making unit being adapted to make an oral model, a tooth mold and implant surgical guide plates subject to said oral cavity simulation diagram; and
a navigator module electrically connected to said control unit and adapted to receive said oral cavity data from said control unit, said navigator module comprising a display unit and a photograph unit, said photograph unit being electrically connected to said display unit and adapted to convert images of the relative position between the oral cavity and the loaded implant surgical guide plates into corresponding electrical image signals and to transmit the electrical image signals to said display unit for generating a picture for enabling the generated picture be overlapped on the displayed simulation picture to help the dentist perform the surgery.

2. The image navigation integrated dental implant system as claimed in claim 1, wherein said control unit is a computer.

3. The image navigation integrated dental implant system as claimed in claim 1, wherein said scan unit has at least one of the functions of full body scan, magnetic resonance imaging (MRI), ultrasound imaging, computer tomography, and intra and extra oral scan.

4. The image navigation integrated dental implant system as claimed in claim 1, wherein said picture-taking unit is a color camera.

5. The image navigation integrated dental implant system as claimed in claim 1, wherein said design unit is a CAD/CAM (computer-aided design/computer-aided manufacturing) design software.

6. The image navigation integrated dental implant system as claimed in claim 1, wherein said mold-making unit is a dental milling machine.

7. The image navigation integrated dental implant system as claimed in claim 1, wherein said display unit is selected from the group of display screen and in-goggle display device.

8. The image navigation integrated dental implant system as claimed in claim 1, wherein said photograph unit is a video camera.
